(19) United States
(12) Patent Application Publication
(10) Pub. No.: US 2009/0326510 A1
(43) Pub. Date: Dec. 31, 2009

(54) Drug Delivery Methods and Systems

(76) Inventors: Paul A. Haefner, Circle Pines, MN (US); Loell Boyce Moon, Ham Lake, MN (US)

Correspondence Address: FISH & RICHARDSON P.C., P.O. BOX 1022, MINNEAPOLIS, MN, 55440-1022, US

(21) Appl. No.: 12/165,328

(22) Filed: Jun. 30, 2008

(51) Int. Cl. A61M 37/00; A61K 49/00; A61P 9/06; A61B 5/02; A61B 5/0402; A61P 7/02; A61P 9/10

(52) U.S. Cl. 604/503; 424/9.2; 600/508; 600/509; 604/66

(57) ABSTRACT

Drug delivery methods and systems that include a determination of whether a cardiac condition is normal or abnormal, so that a drug may be administered in accordance with that determination. In one implementation, a drug delivery device may be controlled to reduce or stop the drug administration when a normal cardiac condition is detected. In another implementation, a patient monitoring device determines the duration that a cardiac condition is normal and provides an output indicative of the determination so that the patient may alter a therapy accordingly.

### TECHNICAL FIELD

This disclosure relates to drug delivery methods and systems, for example, methods and systems that include a determination of whether a cardiac condition is normal or abnormal, so that a drug may be administered in accordance with that determination.

### BACKGROUND

Many people have an abnormal heart condition. One example of abnormal heart conditions is an arrhythmia where the heart beats irregularly. An arrhythmia can be a bradycardia where the heart beats abnormally slow or a tachyarrhythmia where the heart beats abnormally fast. Various different devices and systems are available to detect and address abnormal heart conditions. For example, cardiac stimulation devices may provide electrical therapy to address a detected arrhythmia. In another example, a physician may evaluate a patient's condition, and prescribe drug therapy to address an abnormal condition that the physician determines to be present. In the case of a determined arrhythmia, for example, an antiarrhythmic drug such as Ibutilide may be prescribed to prevent or reduce a patient's arrhythmia. An improvement in the patient's arrhythmic condition may result that a dosage prescribed by a physician becomes unnecessarily strong.

Another example of an arrhythmia condition is atrial fibrillation (AF). AF may be a chronic condition in some patients, or may be non-chronic, in the case of atrial flutter or generally paroxysmal AF. During AF, blood is not pumped effectively from the atria, so it may pool there and clot. If a whole or partial blood clot leaves the heart and becomes lodged in an artery in the brain, a stroke results. Current statistics show that approximately fifteen percent of strokes occur in people with AF.

Anticoagulants such as Warfarin and Heparin thin the blood, making it less prone to clotting. Therefore, these drugs are used to help reduce the risk of stroke in people with AF. Long-term use of Warfarin in patients with AF and other stroke risk factors has been shown to reduce stroke by sixty-eight percent. However, while Warfarin is effective against embolic stroke, its chronic use may have serious side effects which may include potential bleeding problems and ulcers.

### SUMMARY

In one general aspect, a method of regulating drug administration to a patient includes administering a drug at an initial dosage level to a patient via a drug delivery unit. The method also includes monitoring a cardiac signal using a cardiac monitoring unit. The method further includes reducing to a reduced dosage level that is lower than the initial dosage level when the monitored cardiac signal indicates a healthy cardiac sinus rhythm is present for at least a predetermined amount of time.

In various implementations, the method may include one or more of the following features. The method may include administering the drug that may be accomplished by the patient or by a drug delivery system that may be external to the patient or implanted in the patient. The cardiac monitoring unit may be implanted in the patient and may monitor various cardiac signals including an electrocardiogram signal, a blood flow signal, and a blood pressure signal. The drug delivered may include an anticoagulant drug or an antiarrhythmic drug. The initial dosage level may be determined by a physician. The reduced dosage level may be a predetermined minimum level for patient safety that may be selected by a physician.

The method may also include factoring in the ability of blood to properly clot before reducing to the reduced dosage level. The ability of blood to properly clot may be represented by a lab-measured, device-measured or patient-measured International Normalization Ratio (INR), and a desired INR level may be determined by the amount of time that the patient is in a healthy sinus rhythm.

The method may further include resuming the drug administration or increasing the drug dosage when the monitored cardiac signal indicates the absence of a healthy cardiac sinus rhythm for at least a predetermined period of time. The amount of drug dosage increase may be determined by the amount of time the patient is in a healthy sinus rhythm. In some implementations, the dosage increase does not exceed the initial dosage level.

The method may also include tracking the amount of the drug that has been administered to the patient and providing an alert when the amount of the drug administered reaches a predetermined maximum dosage amount. In some implementations, the drug delivery unit and the cardiac monitoring unit are contained within a single housing. In some implementations, the drug delivery unit is contained within a first housing, and the cardiac monitoring unit is contained within a second housing different from the first housing.

In another general aspect, a patient monitoring and notification system includes a cardiac monitoring system having a cardiac condition sensor and a processing unit adapted to receive cardiac signals sensed over a time period by the cardiac condition sensor; determine from the received cardiac signals a time measure indicating a duration of the time period that a cardiac condition is normal; and generate, from the determined duration of the normal cardiac condition, prescriptive information relevant to an on-going therapy being administered to the patient. The system also includes a user notification device adapted to receive the prescriptive information from the cardiac monitoring system, and provide a user output adapted to inform the user as to how the therapy should be administered.

In various implementations, the patient monitoring and notification system may include one or more of the following features. The cardiac monitoring system may be an implantable subcutaneous ECG monitoring system having a first telemetry component to wirelessly transmit information indicative of the determined cardiac condition of normal or abnormal. The notification device may be a component of external equipment having a second telemetry component to receive the wirelessly transmitted information indicative of the determined cardiac condition. The cardiac monitoring system may process a monitored subcutaneous system to evaluate sinus rhythm, and determines a cardiac condition to be normal if a healthy sinus rhythm has been present for a predetermined amount of time. The determination of the cardiac condition being normal or abnormal may involve use of a ratio of an amount of time during which healthy sinus rhythm is present compared to overall time.

In still another general aspect, a drug infusion system includes a cardiac monitoring unit adapted to sense, from a subject, cardiac signals from which a sinus rhythm condition is evidenced if present. The system also includes a drug delivery unit adapted to contain and deliver to the patient an anticoagulant drug. The system further includes a processing and control unit adapted to receive the sensed cardiac signals, perform an assessment of sinus rhythm, and control delivery of the anticoagulant drug by the drug delivery unit based on the assessment of sinus rhythm.

In various implementations, the system may include one or more of the following features. The processing and control unit may be programmable to provide an initial drug dosage level at which the anticoagulant drug is delivered to the patient. The processing and control unit may be adapted to cease delivery of the anticoagulant drug if the processing and control unit determines that the subject is in healthy sinus rhythm for a predetermined amount of time. The processing and control unit may also be adapted to reduce the dosage level at which the anticoagulant drug is delivered if the processing and control unit assesses that a condition exists indicative of a reduced risk of blood clotting from atrial fibrillation. The cardiac monitoring unit may include a cardiac electrical activity sensor or a hemodynamic sensor.

In yet another general aspect, a method for providing anticoagulant therapy to a patient having an atrial fibrillation condition includes controlling, at a programmed initial dosage level, infusion of an anticoagulant drug from a drug delivery unit to a subject. The method also includes monitoring cardiac signals of the subject, and determining from the monitored cardiac signals if there is present an improved condition indicative of a reduced risk of blood clotting in the subject from atrial fibrillation. The method further includes if the improved condition is determined to be present, controlling the infusion of the anticoagulant drug from the drug delivery unit at a reduced dosage level that is lower than the initial dosage level.

In various implementations, the method may include one or more of the following features. The method may include receiving an input providing the programmed initial dosage level. The reduced dosage level may be substantially zero if it is determined that the atrial fibrillation condition has ceased to exist in the subject. The sensed cardiac signals may include electrical signals indicative of cardiac activity or hemodynamic signals indicative of cardiac activity.

The details of one or more implementations are set forth in the accompanying drawings and the description below. Other features, objects, and advantages will be apparent from the description and drawings, and from the claims.

### DESCRIPTION OF DRAWINGS

FIG. 1 is a block diagram of an exemplary drug delivery system;

FIGS. 2A-2C are diagrams that illustrate a variety of different ways that the drug delivery system of FIG. 1 could be implemented;

FIG. 2D is a diagram illustrating a patient monitoring system that provides a notification of a patient cardiac condition.

FIGS. 3A and 3B are flow charts illustrating exemplary methods for regulating drug administration to a patient with abnormal cardiac condition using the drug delivery system shown in FIGS. 2A-2D; and

FIG. 4 is a flow chart illustrating an exemplary method of continued drug administration after implementing the method shown in FIG. 3A.

FIG. 5 is a block diagram of various components that may be used in a device to implement the methods described herein or portions thereof.

Like reference symbols in the various drawings indicate like elements.

### DETAILED DESCRIPTION

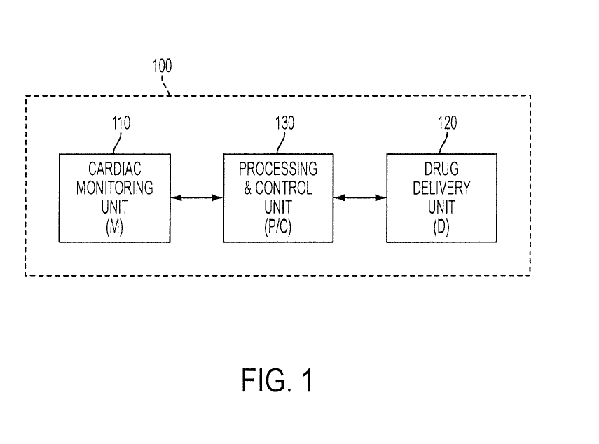

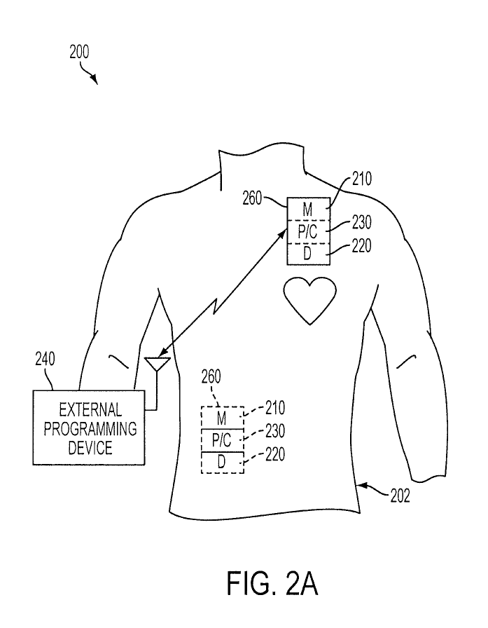

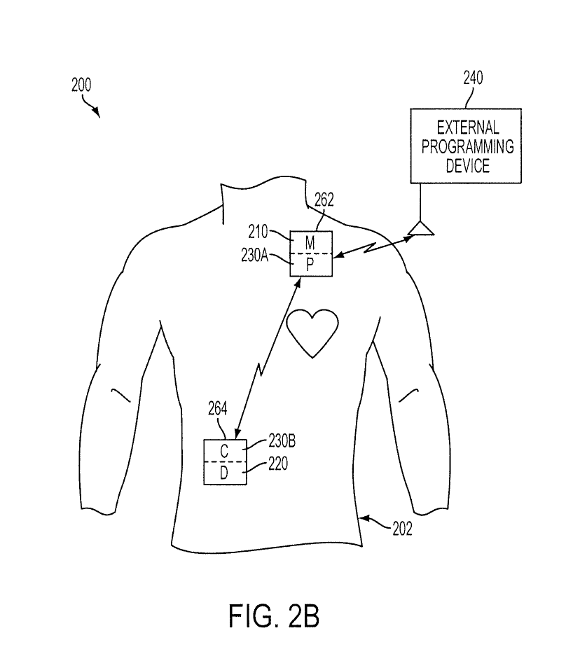

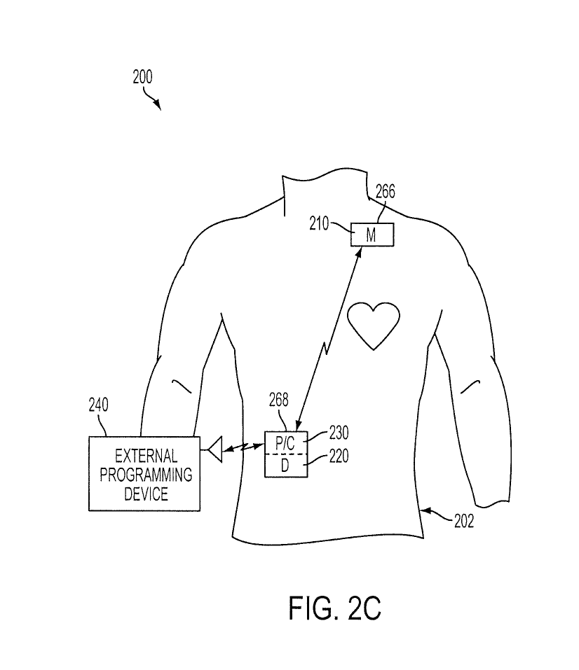

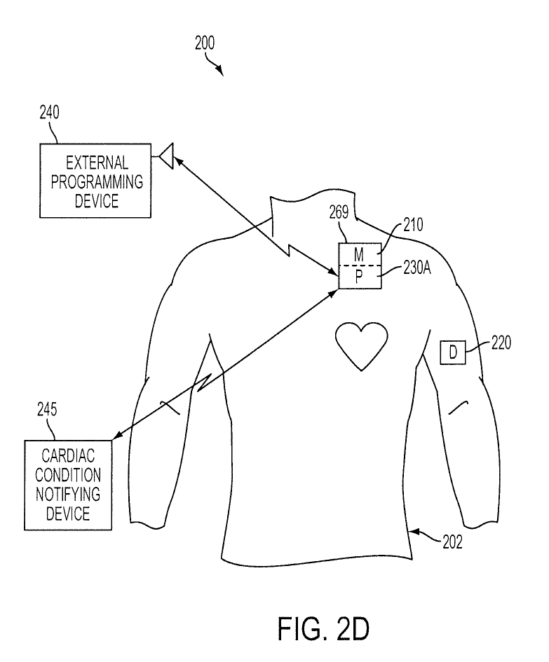

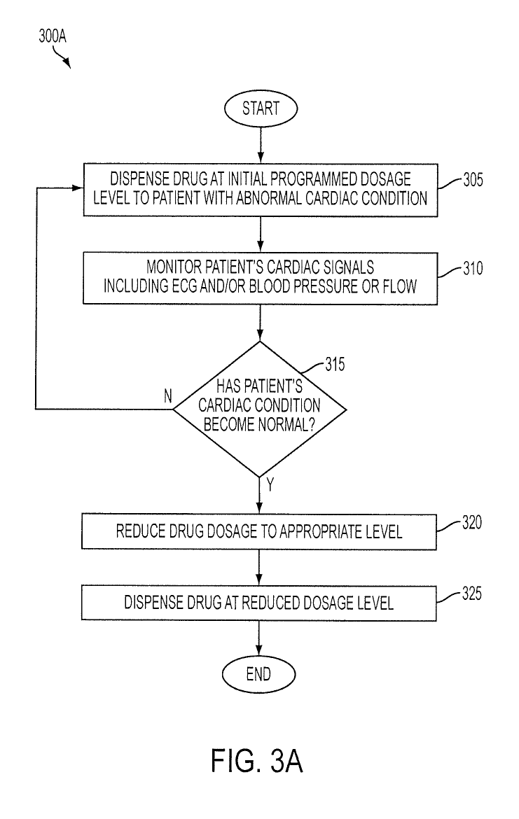

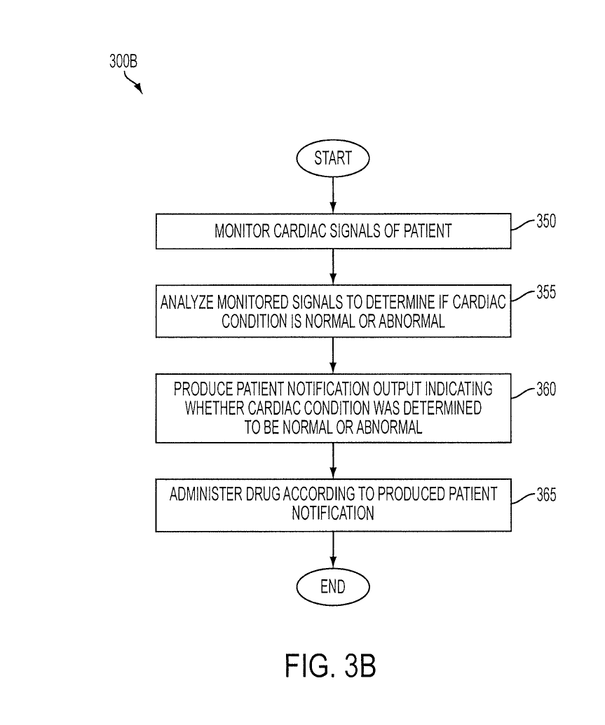

Generally, this document describes systems and methods that include a determination of whether a cardiac condition is normal or abnormal, so that, for example, a drug may be administered in accordance with that determination. In one implementation, shown for example in FIGS. 1, 2A-2C and 3A, a controllable drug delivery unit (for example, a wearable or implantable drug infusion pump) is used, and is controlled in accordance with a determination made as to whether a cardiac condition is normal or abnormal. For example, in a case where it is detected that a cardiac condition is no longer abnormal (i.e., the condition is normal), then the drug delivery device may be controlled to reduce, or perhaps entirely suspend, delivery of the drug. In another implementation, shown for example in FIGS. 2D and 3B, a patient monitoring device monitors and makes a determination as to a duration that a cardiac condition is normal, and provides an output indicative of the determination. In this case, a patient may alter a drug therapy in accordance with the determination of normal or abnormal that is made by the monitoring device.

Referring first to FIG. 1, there is shown a general block diagram of a drug delivery system 100. The drug delivery system 100, generally, includes a cardiac monitoring unit 110 that is adapted to monitor cardiac signals, a drug delivery unit 120 that is adapted to deliver a drug using a pump for example, and a processing and control unit 130 that is adapted to receive and assess monitored cardiac signals and control drug delivery.

Generally, in operation, the drug delivery system 100 may be programmed to perform as follows. Initially, the processing and control unit 130 may be programmed, for example using programming equipment (not shown), to control the drug delivery unit 120 to infuse a drug at an initial dosage level. The cardiac monitoring unit 110 of the system 100 monitors cardiac signals of a subject, for example, an electrocardiogram (ECG) signal. That monitored information may be used by the system 100 to alter the dosage level after the initial programming. In particular, for example, the system 100 may determine that the cardiac activity of the subject is sufficiently satisfactory to reduce the dosage level of the drug being infused. The processing and control unit 130 may be programmed to analyze the monitored signals, and to employ an algorithm that determines when to reduce the dosage level, and by how much. This feature may be useful, for example, in situations where there may be negative consequences to applying a drug when not necessary or in too large of dosage levels. More generally even, it is preferable not to provide a patient a drug unnecessarily.

One example in which the drug delivery system 100 may be particularly useful is in the administration of anticoagulation therapy to a patient with AF. In this example, the processing and control unit 130 may be user programmable to provide an initial dosage level at which an anticoagulant is delivered to the patient by the drug delivery unit 120. Representative examples of anticoagulants include Biotinylated Idraparinux, Heparin, Warfarin, Clopidogrel, and Dipyridamole. The cardiac monitoring unit 110 senses cardiac signals of the patient that may indicate whether an AF condition is present in the patient. Examples of such cardiac signals include ECG and blood pressure or flow. The processing and control unit 130 executes a stored algorithm (i.e., computer program with executable instructions) to receive the sensed cardiac signals and assess the patient's AF condition, and to reduce the dosage level at which the anticoagulant is delivered to the patient or even suspend the delivery of the anticoagulant when the assessment shows the risk of blood clotting from AF is reduced or the patient appears to no longer have an AF condition. This ability to reduce anticoagulant delivery after initial dosage may be a significant improvement on traditional anticoagulation therapies where constant dosage levels may otherwise be carried out for an entire period of time between visits to a physician. During these intervals, the patient's condition may improve, either permanently or for periods of time, and it may be a benefit to reduce the dosage levels when this occurs.

Regarding more details of the basic components of the drug delivery system 100, the cardiac monitoring unit 110 may be any suitable device that is capable of monitoring cardiac signals of a subject. As an example of a cardiac monitoring unit 110 that monitors cardiac electrical activity, the unit 110 may be a sensor that senses an ECG signal and may employ a lead system to do that. The monitoring unit 110 may be an external unit that monitors a skin or surface ECG signal, or may be fully implantable. The lead system includes electrodes that may be provided, for example, on a surface of the unit 110 (e.g., for an implantable unit to monitor a subcutaneous ECG signal), or may extend from the unit 110. The electrical activity may be, for example, a subcutaneous ECG signal, or in an embodiment where the lead system extends into and has electrodes that are positioned within the heart, the electrical activity being monitored may be a cardiac electrogram, also referred to as an Egram or EGM.

Additionally or alternatively to sensing cardiac electrical activity, the cardiac monitoring unit 110 may include a pressure sensor to measure blood pressure or flow. An example of such a pressure sensor is a pressure sensor device that comprises a fluid-filled pressure transmission catheter that transmits a pressure force exerted on a distal tip of the catheter through a pressure transmitting fluid contained within a lumen of the catheter and to a pressure transducer positioned at a proximal end of the pressure transmission catheter. Such a device may be used to measure pressure at a location where the tip of the pressure transmission catheter is positioned, which may be, for example, within a blood vessel of a patient. Other representative examples of catheter-based, blood pressure or flow sensors include Millar catheters and Swann-Ganz catheters. Non-catheter-based pressure sensors may also be used.

The drug delivery unit 120 of the drug delivery system 100 can be any suitable device that is controlled by a control unit and is capable of delivering a drug to a patient. For example, the drug delivery unit 120 can be a drug pump with a reservoir through which the drug is delivered. The drug delivery unit 120 may be an externally worn device, for example clipped to a belt and having an attached infusion set for delivering a drug internally into the patient. Alternatively, the drug delivery unit 120 may be a fully implantable device.

The processing and control unit 130 of the drug delivery system 100 comprises processing circuitry that executes an algorithm to control the receipt of the monitored cardiac signals and the analysis of the signals to determine whether the dosage level of drug infusion should be altered. As one example, the control unit 130 may process the monitored cardiac signals to determine if a healthy cardiac sinus rhythm is present for at least a predetermined period of time, in which case, the dosage level may be reduced, and the control unit 130 may calculate when to reduce the dosage level, and by how much. In addition, for example, the control unit 130 may also, subsequent to causing the dosage level to be reduced, determine that newly monitored cardiac signals show abnormal cardiac activity has returned, and as a result may increase the dosage level, for example, to the level initially programmed, or for example, to a level that is somewhere between the initially programmed level and a present, lower dosage level.

Sinus rhythm is the normal rhythm of the heart originated in the sinoatrial node. Sinus rhythm can be measured by ECG. A healthy sinus rhythm with ECG including normal sinus rhythm, sinus bradycardia and sinus tachycardia is typically characterized by regular rhythm, rate between 40 and 160 beats per minute where rate changes are gradual, P-waves that are upright and have a consistent morphology and precede each QRS complex, PR interval that is from 0.12 to 0.20 seconds, QRS duration that is 0.1 seconds or less, consistent QRS morphology, and the like. Sinus rhythm can also be measured by blood pressure or flow. A healthy sinus rhythm with blood pressure or flow is typically characterized by regular rhythm, consistent amplitude and morphology for a given rate, gradual rate changes, and the like.

In addition to analyzing the monitored cardiac signals, the processing and control unit 130 also controls the operation of the drug delivery unit 120. For example, the processing and control unit 130 can instruct the drug delivery unit 120 to dispense to a patient a drug at an initial dosage level that is determined by a physician to treat the patient's abnormal cardiac condition. The processing and control unit 130 can also command the drug delivery unit 120 to reduce the drug dosage level, or stop the drug administration entirely, when the unit 130 determines that the monitored cardiac signals indicate a healthy cardiac sinus rhythm for at least a predetermined period of time.

FIGS. 2A-2D illustrate a variety of different ways that the drug delivery system 100 of FIG. 1 could be implemented. FIG. 2A illustrates a drug delivery system 200 where the cardiac monitoring unit 210, the drug delivery unit 220 and the processing and control unit 230 are all contained within a single housing 260. The system 200 also includes an external programming device 240 that communicates, for example wirelessly, with the processing and control unit 230. As shown in FIG. 2A, the housing 205 can be implanted in the body of a patient 202 or worn by the patient 202, and in particular implanted subcutaneously or worn in a pectoral region of the patient's body 202. Alternatively, the housing 205 can be implanted subcutaneously or worn in an abdominal region of the patient's body 200 with the sensing electrodes (not shown) of the monitoring unit 210 placed in the heart. Such positioning is useful, for example, to enable the monitoring unit 210 to monitor, for example, an electrocardiogram (ECG) signal of the body's heart. In other embodiments, however, the monitoring unit 210 may monitor blood pressure or flow. Communication links (not shown) such as bus links allow signal transmissions between the processing and control unit 230 and the cardiac monitoring unit 210 and the drug delivery unit 220. The external programming device 240 can be any suitable device where a user (e.g., patient or physician) can input various commands to program the processing and control unit 230.

FIG. 2B illustrates a drug delivery system 200 where the cardiac monitoring unit 210 and the processing component 230A of the processing and control unit 230 are contained within one housing 262 while the drug delivery unit 220 and the control component 230B of the processing and control unit 230 are contained within a different housing 264. The system 200 also includes an external programming device 240 that communicates, for example wirelessly, with the processing component 230A of the unit 230. As shown in FIG. 2B, both housings 262, 264 can be implanted in the body of a patient 202 or worn by the patient 202, and in particular the housing 262 implanted subcutaneously or worn in a pectoral region of the patient's body 202 and the housing 264 implanted subcutaneously or worn in an abdominal region of the patient's body 202. Communication links (not shown) such as bus links allow signal transmission between the processing component 230A of the unit 230 and the cardiac monitoring unit 210 and between the control component 230B of the unit 230 and the drug delivery unit 220. A telemetry link such as a wireless data transmission link allows communication between the processing component 230A and the control component 230B of the unit 230.

FIG. 2C illustrates a drug delivery system 200 where the cardiac monitoring unit 210 is contained within one housing 266 while the drug delivery unit 220 and the processing and control unit 230 are contained within a different housing 268. The system 200 also includes an external programming device 240 that communicates, for example wirelessly, with the processing and control unit 230. As shown in FIG. 2C, both housings 266, 268 can be implanted in the body of a patient 202 or worn by the patient 202, and in particular the housing 266 implanted subcutaneously or worn in a pectoral region of the patient's body 202 and the housing 268 implanted subcutaneously or worn in an abdominal region of the patient's body 202. A communication link (not shown) such as a bus link allows signal transmission between the processing and control unit 230 and the drug delivery unit 220. A telemetry link such as a wireless data transmission link allows communication between the processing and control unit 230 and the cardiac monitoring unit 210.

FIG. 2D illustrates a patient monitoring and notification system 280 where the cardiac monitoring unit 210 and a processing component 230A are contained within one housing 269. The patient monitoring and notification system 280 also includes an external programming device 240 that communicates, for example wirelessly, with the processing component 230A. The system 280 further includes a cardiac condition notifying device 245. The notifying device 245 can be any suitable device that is capable of providing notice to a patient or physician or caregiver about the status of the patient's cardiac condition (e.g., whether the cardiac condition is normal or abnormal). For example, the notifying device 245 may be an indicator that is wearable by a patient or a patient bedside monitor that a physician or caregiver checks regularly. The drug delivery unit 220 can be a drug patch that can be adhered, for example, to an arm of a patient 202. As shown in FIG. 2D, the housing 269 can be implanted in the body of a patient 202 or worn by the patient 202, and in particular implanted subcutaneously or worn in a pectoral region of the patient body 202. The processing component 230A communicates with the notifying device 245 via a telemetry link such as a wireless data transmission link. If the processing component 230A determines that the patient's cardiac condition is sufficiently satisfactory to reduce the drug dosage level, the component 230A sends a message to the notifying device 245, reminding the patient who wears the notifying device 240 or a physician or caregiver who receives the message to remove the previously attached drug patch or to replace the previously attached drug patch with a new patch that has a lower drug dosage level or a new drug with less pharmaceutical effect.

FIGS. 3A and 3B are flow charts illustrating exemplary methods 300A, 300B that include a determination of whether a cardiac condition is normal or abnormal. The method 300A shown in FIG. 3A may be performed, for example, by the systems 200 shown in FIGS. 1 and 2-2C, and the method 300B shown in FIG. 3B may be performed, for example, by the system 280 shown in FIG. 2D.

Referring first to FIG. 3A, at step 305, the processing and control unit 230 of the system 200 instructs the drug delivery unit 220 of the system 280 to infuse to a patient with abnormal cardiac condition a drug at an initial programmed dosage level that is determined by a physician to treat the patient's abnormal cardiac condition. In one implementation, the drug administered includes an antiarrhythmic drug. Representative example of antiarrhythmic drugs include Digoxin, Amiodarone, Dronedarone, Sotalol and Ibutilide. In another implementation, the drug administered includes an anticoagulant drug. Representative examples of anticoagulant drugs include Biotinylated Idraparinux, Heparin, Warfarin, Clopidogrel, and Dipyridamole. In one implementation, the drug is delivered subcutaneously. In another implementation, the drug is delivered intravenously. In still another implementation, the drug is delivered orally.

At step 310, the cardiac monitoring unit 210 of the system 200 monitors cardiac signals of the patient including the ECG, blood pressure or flow, or the like. In one implementation, the monitoring unit 210 monitors the ECG. In another implementation, the monitoring unit 210 monitors the blood pressure or flow. In still another implementation, the monitoring unit 210 monitors both the ECG and the blood pressure or flow.

At step 315, the processing and control unit 230 executes a stored algorithm to receive the monitored cardiac signals from the cardiac monitoring unit 210 and analyze the signals to assess whether the patient's cardiac condition has become normal. In one implementation, the monitoring unit 210 transmits the monitored cardiac signals to the processing and control unit 230 via a communication link such as a bus link. In another implementation, the monitoring unit 210 transmits the monitored cardiac signals to the processing and control unit 230 via a telemetry link 260 such as a wireless data transmission link.

If at step 315 the processing and control unit 230 determines that the patient's cardiac condition has been normal for at least a predetermined period of time, the unit 230 at step 320 reduces the drug delivery dosage to an appropriate level that may be determined by a physician and at step 325 commands the drug delivery unit 220 to dispense the dosage at the reduced dosage level. If at step 315 the processing and control unit 230 determines that the patient's cardiac condition is still abnormal, steps 305-325 are repeated until the unit 230 determines that the patient's cardiac condition has been normal for at least a predetermined period of time.

In one implementation where an anticoagulant drug is administered to treat a patient with an AF condition, the processing and control unit 230 may decrease the initial anticoagulant drug dosage level to a proper lower dosage level that may be prescribed by a physician when the unit 230 detects a condition that indicates a reduced risk of blood clotting from AF. The tendency of blood to clot can be represented by international normalized ratio (INR) which measures the time it takes for blood to clot and compares the time measured to an average normal time. An INR test can be performed in a laboratory or near a patient such as at the patient's home. The higher the INR, the longer it takes blood to clot. In healthy people, the INR is about 1.0. For AF patients on anticoagulants, the INR typically should be between 2.0 and 3.0. The risk of blood clotting from a patient's AF condition may thus be considered to be reduced if the patient has been taking an anticoagulant such as Warfarin and maintaining 2 to 3 INR for at least a predetermined period of time.

In another implementation where an anticoagulant drug is administered to treat a patient with an AF condition, the processing and control unit 230 may suspend the anticoagulant drug administration entirely when the unit 230 detects that the patient's AF condition has ceased to exist. A patient's AF condition may have ceased to exist if the patient has exhibited a healthy cardiac sinus rhythm for at least a predetermined period of time. A healthy sinus rhythm with ECG including normal sinus rhythm, sinus bradycardia and sinus tachycardia is typically characterized by regular rhythm, rate between 40 and 160 BPM where rate changes are gradual, P-waves that are upright and have a consistent morphology and precede each QRS complex, PR interval that is from 0.12 to 0.20 seconds, QRS duration that is 0.1 seconds or less, consistent QRS morphology, and the like. A healthy sinus rhythm with blood pressure or flow is typically characterized by regular rhythm, consistent amplitude and morphology for a given rate, gradual rate changes, and the like.

In one implementation, the drug delivery unit 220 can measure the amount of the drug that has been delivered to the patient. If the amount delivered reaches or exceeds a predetermined maximum amount, the drug delivery unit 220 provides an alert to the patient or a physician/caregiver to stop the drug administration. In another implementation, the drug delivery unit 220 can measure the amount of the drug that remains in its drug reservoir. If the remaining drug amount is below a predetermined level, the drug delivery unit 220 informs the patient or a physician/caregiver to refill the drug reservoir.

Referring to FIG. 3B, at step 350, the cardiac monitoring unit 210 of the system 200 monitors cardiac signals of the patient including the ECG, blood pressure or flow, or the like. At step 355, the processing and control unit 230 of the system 200 analyzes the monitored cardiac signals to determine whether the patient's cardiac condition is normal or abnormal. A patient has a normal cardiac condition if the patient exhibits a healthy cardiac sinus rhythm for at least a predetermined period of time. At step 360, the cardiac condition notifying device 245 of the system 200 produces a message to the patient or a physician/caregiver, indicating whether the patient's cardiac condition was determined to be normal or abnormal. Based on this notification message, the patient or physician/caregiver at step 365 can administer an appropriate drug dosage accordingly. In one implementation, the patient or physician/caregiver can remove a previously attached drug patch or to replace the previously attached drug patch with a new patch that has a lower drug dosage level or a new drug with less pharmaceutical effect.

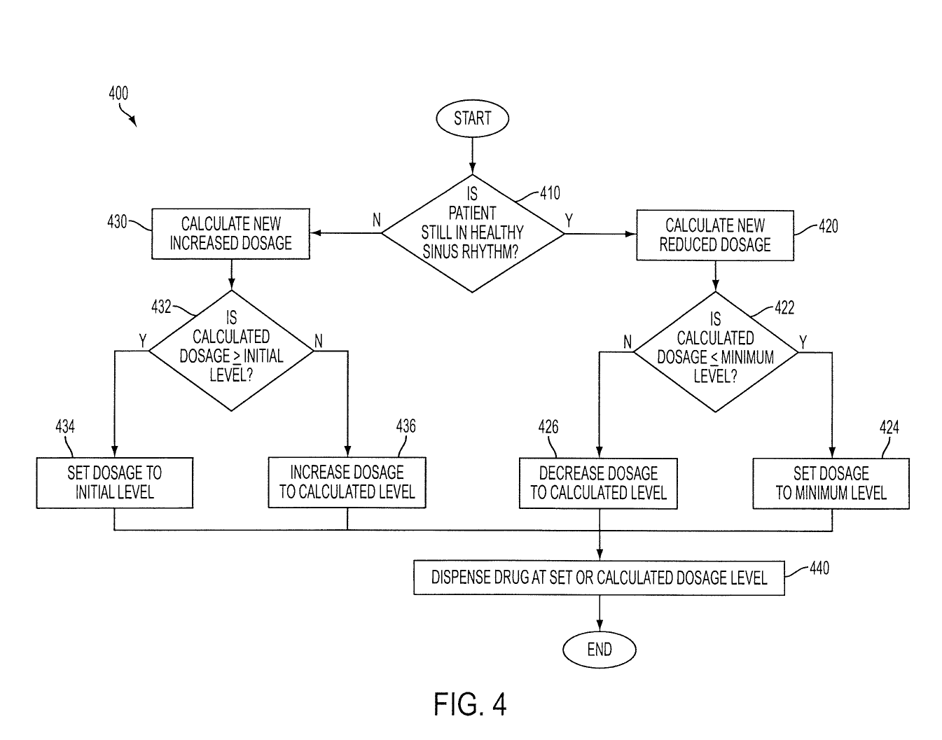

FIG. 4 is a flow chart illustrating an exemplary method 400 of continued drug administration after implementing the method 300A of FIG. 3A. At step 410, the processing and control unit 230 assesses whether the patient is still in a healthy sinus rhythm condition for at least a predetermined period of time at the current drug dosage level. If so, the unit 230 at step 420 calculates a new reduced dosage level at which the drug should be delivered. At step 422, the unit 230 determines whether the calculated reduced dosage level has already reached or gone below a predetermined minimum dosage level that may be selected by a physician. If so, the unit 230 at step 424 sets the drug dosage to the predetermined minimum level and then at step 440 signals the drug delivery unit 220 to dispense the drug at the minimum dosage level. In one implementation, the predetermined minimum dosage level is a dosage level below which the patient with AF may suffer strokes as a result of an under-administration of an anticoagulant drug. In another implementation, the predetermined minimum dosage level is substantially zero.

If at step 422 the processing and control unit 230 determines that the reduced drug dosage level that is calculated at step 420 is still above the predetermined minimum level, the unit 230 at step 426 decreases the drug dosage to the level calculated at step 420 and then at step 440 instructs the drug delivery unit 220 to infuse the drug at the calculated reduced dosage level.

If at step 410 the processing and control unit 230 detects that the patient's cardiac condition is no longer normal, the unit 230 at step 430 calculates a new increased dosage level at which the drug should be delivered to remedy the patient's recurring abnormal cardiac condition. At step 432, the unit 230 determines whether the increased dosage level that is calculated at step 430 has already reached or exceeded the initial dosage level that may be programmed by a physician. If so, the unit 230 at step 434 sets the drug dosage to the initial programmed level and then at step 440 commands the drug delivery unit 220 to deliver the drug at the initial dosage level. Otherwise, the unit 230 increases the drug dosage to the level calculated at step 430 and then at step 440 signals the unit 220 to dispense the drug at the calculated increased dosage level.

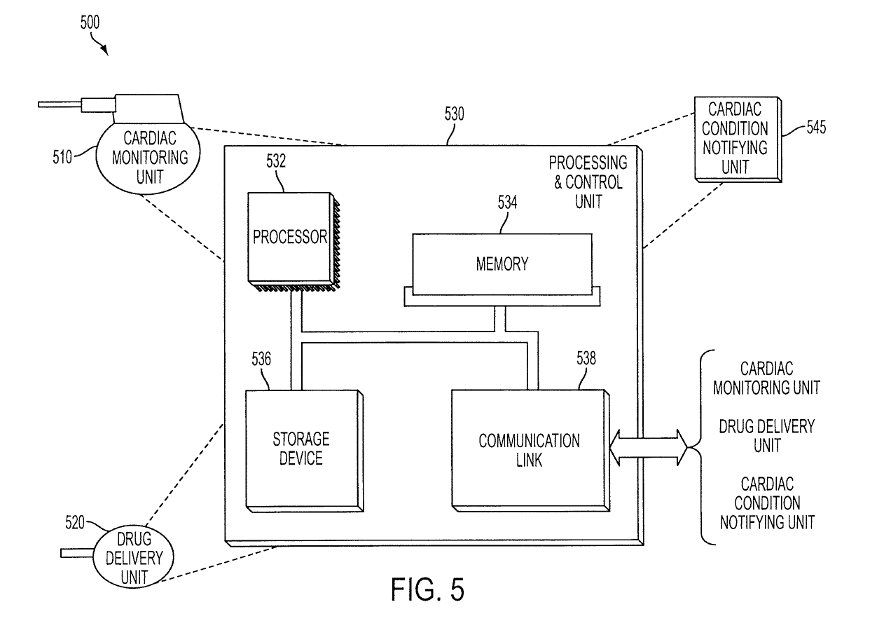

FIG. 5 is a block diagram illustrating various components that may be incorporated into a device 500 to perform the previously described methods. In some implementations, the device 500 can implement any of the methods described herein, or any portion of the methods. In some cases, the device 500 can cooperate with one or more other devices (whether implanted or external) to implement the methods discussed herein. The device 500 may include a cardiac monitoring unit 510, a drug delivery unit 520, a processing and control unit 530, and a cardiac condition notifying unit. The processing and control unit 530 includes a processor 532, a memory 534, a storage device 536, and a communication link 538. Each of the components 532, 534, 536, and 538 are interconnected using bus links.

The cardiac monitoring unit 510 is capable of sensing a patient's cardiac signals over a predetermined number of heartbeats or for a predetermined time interval and transmitting the sensed signals to the processing and control unit 530 for further processing. For example, the unit 510 can sense and transmit an ECG signal, or a hemodynamic signal. In some implementations, the unit 510 may sense and transmit multiple cardiac signals, such as both an electrical signal and a hemodynamic signal, either or both of which may be analyzed independently or cooperatively to determine cardiac conditions.

In some implementations, the monitoring unit 510 includes one or more leads (not shown), which may be configured for positioning inside or outside of a patient's heart. The one or more leads can include one or more electrodes that can sense cardiac signals. In some implementations, the one or more leads are intracardiac leads; in some implementations, the one or more leads are configured for subcutaneous positioning within a patient; in some implementations, at least one intracardiac lead and at least one subcutaneous lead are included. In some implementations, the one or more leads may be replaced or supplemented with one or more sensors or ports configured to sense a hemodynamic signal. Some implementations may include one or more catheters that may facilitate hemodynamic measurements at a distance from the device. For example, a pressure transmission catheter may be used to sense a body pressure and refer the pressure to a pressure transducer, which may be housed within the body of the monitoring unit 510 or in a separate housing, in which case the pressure information may be communicated to the monitoring unit by wired or wireless communication link. Various combinations of leads and electrodes are possible. As one example, the monitoring unit 510 may include a single lead with a single electrode, and may include a second electrode on the housing of the unit 510. As another example, a lead may include two or more electrodes, or the housing may include two or more electrodes. Leadless implantable devices are also contemplated, where an exterior surface or the device includes electrodes and/or sensor(s) to make the measurements discussed herein.

In some implementations, the monitoring unit 510 includes a transceiver (not shown). The transceiver may include a transmitter and a receiver, and may communicate wirelessly with an external (or implanted) device, such as the processing and control unit 530, using an antenna. In some implementations, the transceiver can be configured to receive command signals. For example, the receiver can receive a command that instructs the monitoring unit 510 to record a cardiac signal. In other implementations, the transceiver can receive a command signal indicating that the monitoring unit 510 should record and transmit a cardiac signal on a specified periodic basis, such as hourly, daily, or weekly. In other implementations, the transceiver can receive a command signal that instructs the monitoring unit 510 to measure a cardiac signal and transmit the signal without storing it within the unit 510, including continuous measurement and transmit in some implementations.

The drug delivery unit 520 is capable of delivering a drug to a patient under the control of the processing and control unit 530. In some implementations, the drug delivery unit 520 is a drug pump with a reservoir through which the drug is delivered. The drug delivery unit 520 may be an externally worn device, for example clipped to a belt and having an attached infusion set for delivering a drug internally into the patient. Alternatively, the drug delivery unit 120 may be a fully implantable device. In some implementations, the drug delivery unit 520 delivers the drug subcutaneously. In some implementations, the drug delivery unit 520 delivers the drug intravenously.

In one implementation, the processing and control unit 530 is capable of receiving the cardiac signals measured by the monitoring unit 520, assessing the monitored signals to determine whether a patient's cardiac condition is normal or abnormal, and controlling the drug dosage delivered by the drug delivery unit 520 in accordance with that determination. The processor 532 of the processing and control unit 530 is capable of receiving and processing instructions stored in the memory 534 of the unit 530 to perform the methods disclosed herein or portions thereof. The processor 532 can be any suitable processors for the execution of instructions including both general and special purpose microprocessors, and sole processor or multiple processors. The processor 532 can execute instructions to cause the unit 530 to receive the monitored cardiac signal from the monitoring unit 510 via the communication link 538 of the unit 530. The processor 532 can also execute instructions to cause the unit 530 to process the received cardiac signals to assess whether a patient's cardiac condition is normal or abnormal. The processor 532 can further execute instructions to cause the unit 530 to send via the communication link 538 a control signal to the drug delivery unit 520. Based on the assessment, the signal can instruct the unit 520 to reduce the dosage administered or stop the drug administration completely if the patient's cardiac condition is normal, or to increase the drug dosage level or resume drug delivery if the patient's cardiac condition is abnormal. In some implementations, processor 532 and the memory 534 may be implemented in a programmable device, such as a programmable logical device (PLD, e.g. an FPGA) or application specific integrated circuit (ASIC).

In another implementation, the processing and control unit 530 is capable of receiving the cardiac signals that the monitoring unit 520 has sensed over a time period, determining from the received signals a time measure that indicates a duration of the time period that a cardiac condition is normal, and generating from the determined time measure prescriptive information that is relevant to a therapy being administered to a patient. The processor 532 of the unit 530 can execute instructions to cause the unit 530 to receive the cardiac signals that the monitoring unit 520 has measured over a time period via the communication link 538 of the unit 530. The processor 532 can also execute instructions to cause the unit 530 to process the received cardiac signals to determine the duration of the time period that a cardiac condition is normal. The processor 532 can further execute instructions to cause the unit 530 to generate based on the determined duration prescriptive information that is relevant to an on-going patient therapy and send via the communication link 538 the prescriptive information to the cardiac condition notifying unit 545. Based on the information received, the unit 545 can provide a user output to inform the user as how the therapy should be administered. The cardiac condition monitoring unit The notifying device 245 can be any suitable device that is capable of providing notice to a patient or physician or caregiver about the status of the patient's cardiac condition (e.g., whether the cardiac condition is normal or abnormal). For example, the notifying device 245 may be an indicator that is wearable by a patient or a patient bedside monitor that a physician or caregiver checks regularly.

The memory 534 can be any suitable memory that is capable of storing information. For example, the memory 534 can be a read-only memory or a random access memory or both. The communication link 538 can be any suitable link that is capable of transmitting signal data. In some implementations, the communication link 538 includes a telemetry component that can transmit or receive data wirelessly over an antenna. In some implementations, the communication link 538 includes a bus link that provides interconnectivity between the processing and control unit 530 and the monitoring unit 510 and the drug delivery unit 520. The storage device 536 of the unit 530 can be any suitable storage device that is capable of providing mass storage such data files. For example, the storage device 534 can be a computer-readable medium such a hard disk device or an optical disk device.

A number of embodiments of the invention have been described. Nevertheless, it will be understood that various modifications may be made without departing from the spirit and scope of the invention. Accordingly, other embodiments are within the scope of the following claims.

What is claimed is:

1. A method of regulating drug administration to a patient, comprising:
administering a drug at an initial dosage level to a patient via a drug delivery unit;
monitoring a cardiac signal using a cardiac monitoring unit; and
reducing to a reduced dosage level that is lower than the initial dosage level when the monitored cardiac signal indicates a healthy cardiac sinus rhythm is present for at least a predetermined amount of time.

2. The method of claim 1, wherein administering the drug is accomplished by the patient.

3. The method of claim 1, wherein administering the drug is accomplished by a drug delivery system.

4. The method of claim 3, wherein the drug delivery system is external to the patient.

5. The method of claim 3, wherein the drug delivery system is implanted in the patient.

6. The method of claim 1, wherein the cardiac monitoring unit is implanted in the patient.

7. The method of claim 1, wherein the drug comprises an anticoagulant.

8. The method of claim 1, wherein the drug comprises an antiarrhythmic drug.

9. The method of claim 1, wherein the initial dosage level is determined by a physician.

10. The method of claim 1, wherein the cardiac signal is an electrocardiogram signal.

11. The method of claim 1, wherein the cardiac signal is a blood flow signal.

12. The method of claim 1, wherein the cardiac signal is a blood pressure signal.

13. The method of claim 1, wherein the reduced dosage level is a predetermined minimum level for patient safety.

14. The method of claim 13, wherein the predetermined minimum dosage level is physician selectable.

15. The method of claim 1, further comprising factoring in the ability of blood to properly clot before reducing to the reduced dosage level.

16. The method of claim 15, wherein the ability of the blood to properly clot is represented by a lab-measured, device-measured or patient-measured International Normalization Ratio (INR).

17. The method of claim 16, further comprising determining a desired INR level by the amount of time that the patient is in a healthy sinus rhythm.

18. The method of claim 1, further comprising resuming the drug administration or increasing the drug dosage when the monitored cardiac signal indicates the absence of a healthy cardiac sinus rhythm for at least a predetermined period of time.

19. The method of claim 18, wherein the amount of drug dosage increase is determined by the amount of time the patient is in a healthy sinus rhythm.

20. The method of claim 18, wherein the increased dosage does not exceed the initial dosage level.

21. The method of claim 1, further comprising tracking the amount of the drug that has been administered to the patient.

22. The method of claim 21, further comprising providing an alert when the amount of the drug administered reaches a predetermined maximum dosage amount.

23. The method of claim 1, wherein the drug delivery unit and the cardiac monitoring unit are contained within a single housing.

24. The method of claim 1, wherein the drug delivery unit is contained within a first housing, and the cardiac monitoring unit is contained within a second housing different from the first housing.

25. A patient monitoring and notification system, comprising:
a cardiac monitoring system comprising a cardiac condition sensor and a processing unit adapted to receive cardiac signals sensed over a time period by the cardiac condition sensor; determine from the received cardiac signals a time measure indicating a duration of the time period that a cardiac condition is normal; and generate, from the determined duration of the normal cardiac condition, prescriptive information relevant to an on-going therapy being administered to the patient; and
a user notification device adapted to receive the prescriptive information from the cardiac monitoring system, and provide a user output adapted to inform the user as to how the therapy should be administered.

26. The patient monitoring and notification system of claim 25, wherein the cardiac monitoring system is an implantable subcutaneous ECG monitoring system comprising a first telemetry component to wirelessly transmit information indicative of the determined cardiac condition of normal or abnormal; and the notification device is a component of external equipment comprising a second telemetry component to receive the wirelessly transmitted information indicative of the determined cardiac condition.

27. The patient monitoring and notification system of claim 26, wherein the cardiac monitoring system processes a monitored subcutaneous system to evaluate sinus rhythm, and determines a cardiac condition to be normal if a healthy sinus rhythm has been present for a predetermined amount of time.

28. The patient monitoring and notification system of claim 27, wherein the determination of the cardiac condition being normal or abnormal involves use of a ratio of an amount of time during which healthy sinus rhythm is present compared to overall time.

29. A drug infusion system comprising:
a cardiac monitoring unit adapted to sense, from a subject, cardiac signals from which a sinus rhythm condition is evidenced if present;
a drug delivery unit adapted to contain and deliver to the patient an anticoagulant drug; and
a processing and control unit adapted to receive the sensed cardiac signals, perform an assessment of sinus rhythm, and control delivery of the anticoagulant drug by the drug delivery unit based on the assessment of sinus rhythm.

30. The drug infusion system of claim 29, wherein the processing and control unit is programmable to provide an initial drug dosage level at which the anticoagulant drug is delivered to the patient.

31. The drug infusion system of claim 29, wherein the processing and control unit is adapted to cease delivery of the anticoagulant drug if the processing and control unit determines that the subject is in healthy sinus rhythm for a predetermined amount of time.

32. The drug infusion system of claim 29, wherein the processing and control unit is adapted to reduce the dosage level at which the anticoagulant drug is delivered if the processing and control unit assesses that a condition exists indicative of a reduced risk of blood clotting from atrial fibrillation.

33. The drug infusion system of claim 29, wherein the cardiac monitoring unit comprises a cardiac electrical activity sensor.

34. The drug infusion system of claim 29, wherein the cardiac monitoring unit comprises a hemodynamic sensor.

35. A method for providing anticoagulant therapy to a patient having an atrial fibrillation condition, the method comprising:
controlling, at a programmed initial dosage level, infusion of an anticoagulant drug from a drug delivery unit to a subject;
monitoring cardiac signals of the subject, and determining from the monitored cardiac signals if there is present an improved condition indicative of a reduced risk of blood clotting in the subject from atrial fibrillation; and
if the improved condition is determined to be present, controlling the infusion of the anticoagulant drug from the drug delivery unit at a reduced dosage level that is lower than the initial dosage level.

36. The method of claim 35, further comprising receiving an input providing the programmed initial dosage level.

37. The method of claim 35, wherein the reduced dosage level is zero if it is determined that the atrial fibrillation condition has ceased to exist in the subject.

38. The method of claim 35, wherein the sensed cardiac signals comprise electrical signals indicative of cardiac activity.

39. The method of claim 35, wherein the sensed cardiac signals comprise hemodynamic signals indicative of cardiac activity.
